(19) United States
(12) Patent Application Publication
(10) Pub. No.: US 2004/0225164 A1
(43) Pub. Date: Nov. 11, 2004

(54) Method for treating ethane

(75) Inventors: Joe D. Allison, Ponca City, OK (US); Timothy J. Eggeman, Lakewood, CO (US); Steven R. McDonald, Ponca City, OK (US); Kent A. Pennybaker, Lawrence, KS (US)

Correspondence Address: CONOCOPHILIPS COMPANY, P.O. BOX 2443, BARTLESVILLE, OK, 74004, US

(73) Assignee: ConocoPhillips Company, Houston, TX (US)

(21) Appl. No.: 10/436,685

(22) Filed: May 9, 2003

(51) Int. Cl. C07C 29/48

(52) U.S. Cl. 568/910

(57) ABSTRACT

Methods are disclosed for converting ethane to ethanol through a multi-step process with ethylene as an intermediate. Methods are also disclosed for facilitating the transportation, purification or other treatment of ethylene using a chemical conversion to ethanol and reconversion to ethylene. Methods are also disclosed for converting ethane to ethylene using ethanol as a temporary intermediate to minimize purification, transportation and/or other treatment costs.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

1. Technical Field of the Invention

The present invention is directed towards downstream treatment and purification of ethylene generated by dehydrogenation of ethane. More particularly, the present invention is directed toward a process for purifying and transporting ethylene derived from ethane by hydrating the ethylene to form ethanol.

2. Description of Related Art

There is currently a significant interest in various types of hydrocarbon processing reactions. One such class of reactions involves the chemical conversion of natural gas, a relatively low value reactant, to higher value products. Natural gas comprises several components, including alkanes. Alkanes are saturated hydrocarbons i.e., compounds consisting of hydrogen (H) and carbon (C)—whose molecules contain carbon atoms linked together by single bonds. The principal alkane in natural gas is methane; however, significant quantities of longer-chain alkanes such as ethane (CH₃CH₃) may also be present. Unlike long-chain alkanes, ethane is gaseous under ambient conditions.

The interest in the chemical conversion of the methane and ethane in natural gas stems from a variety of factors. First, vast reserves of natural gas have been found in remote areas where no local market exists. There is great incentive to exploit these natural gas formations because natural gas is predicted to outlast liquid oil reserves by a significant margin. Unfortunately, though, the transportation costs for methane and ethane are generally high, primarily because of the extremely low temperatures needed to liquefy these highly volatile gases for transport. Consequently, there is considerable interest in techniques for converting methane and ethane to higher value, more easily transported products at the remote site.

Several hydrocarbon processing techniques are currently being investigated for the chemical conversion of lower alkanes. One such technique involves the conversion of methane to higher chain-length alkanes that are liquid or solid at room temperature. This conversion of methane to higher hydrocarbons is typically carried out in two steps. In the first step, methane is partially oxidized to produce a mixture of carbon monoxide and hydrogen known as synthesis gas or syngas. In a second step, the syngas is converted to liquid and solid hydrocarbons using the Fischer-Tropsch process. This method allows the conversion of synthesis gas into liquid hydrocarbon fuels and solid hydrocarbon waxes. The high molecular weight waxes thus produced provide an ideal feedstock for hydrocracking, which ultimately yields high quality jet fuel and superior high cetane value diesel fuel blending components.

Another important class of hydrocarbon processing reactions are dehydrogenation reactions. In a dehydrogenation process, alkanes can be dehydrogenated to produce alkenes. Alkenes, also commonly called olefins, are unsaturated hydrocarbons whose molecules contain one or more pairs of carbon atoms linked together by a double bond. Generally, olefin molecules are represented by the chemical formula R′CH═CHR, where C is a carbon atom, H is a hydrogen atom, and R and R′ are each an atom or a pendant molecular group of varying composition. One example of a dehydrogenation reaction is the conversion of ethane to ethylene [1]:

C₂H₆+Heat→C₂H₄+H₂ [1].

The non-oxidative dehydrogenation of ethane to ethylene is endothermic, meaning that heat energy must be supplied to drive the reaction.

Alkenes such as ethylene are typically higher value chemicals than their corresponding alkanes. This is true, in part, because alkenes are important feedstocks for producing various commercially useful materials such as detergents, high-octane gasolines, pharmaceutical products, plastics, synthetic rubbers and viscosity additives. Ethylene, a raw material in the production of polyethylene, is the one of the most abundantly produced chemicals in the United States. Consequently, cost-effective methods for producing ethylene are of great commercial interest.

Traditionally, the dehydrogenation of hydrocarbons has been carried out using fluid catalytic cracking (FCC), a non-oxidative dehydrogenation process, or steam cracking. Heavy alkenes, those containing five or more carbon atoms, are typically produced by FCC; in contrast, light olefins, those containing two to four carbon atoms, are typically produced by steam cracking. FCC and steam cracking have several drawbacks. First, both processes are highly endothermic requiring input of energy. In addition, some of the ethane reactant is lost as carbon deposits known as coke. These carbon deposits not only decrease yields but also deactivate the catalysts used in the FCC process. The costs associated with heating, yield loss and catalyst regeneration render these processes expensive even without regard to catalyst cost.

Recently, there has been increased interest in oxidative dehydrogenation (ODH) as an alternative to FCC and steam cracking. In ODH, alkanes are dehydrogenated in the presence of an oxidant such as oxygen, typically in a short contact time reactor containing an ODH catalyst. ODH can be used, for example, to convert ethane and oxygen to ethylene and water [2]:

C₂H₆+1/2O₂→C₂H₄+H₂O+Heat [2].

Thus, ODH provides an alternative chemical route to generating ethylene from ethane. Unlike non-oxidative dehydrogenation, however, ODH is exothermic, meaning that it produces rather than requires heat energy.

Although ODH involves the use of a catalyst, which is referred to herein as an ODH catalyst, and is therefore literally a catalytic dehydrogenation, ODH is distinct from what is normally called “catalytic dehydrogenation” in that the former involves the use of an oxidant and the latter does not. ODH is attractive because the capital costs for olefin production via ODH are significantly less than with the traditional processes. ODH, unlike traditional FCC and steam cracking, can be employed using simple fixed bed reactor designs and high volume throughput.

More important, however, is the fact that ODH is exothermic. The net ODH reaction can be viewed as two separate processes: an endothermic dehydrogenation of an alkane coupled with a strongly exothermic combustion of hydrogen, as depicted in [3]:

$$\frac{\begin{matrix}{C}_{2} {H}_{6} + Heat \to {C}_{2} {H}_{4} + {H}_{2} \\ 1 \text{/} 2 {O}_{2} + {H}_{2} \to {H}_{2} O + Heat\end{matrix}}{{C}_{2} {H}_{6} + 1 \text{/} 2 {O}_{2} \to {C}_{2} {H}_{4} + {H}_{2} O + Heat} . \qquad [ 3 ]$$

Energy savings over traditional, endothermic processes can be especially significant if the heat produced in the ODH process is recaptured and recycled.

Unfortunately, although ODH offers the possibility of cost-effective ethylene production, the prohibitive cost for ethane and ethylene transportation has limited interest in the exploitation of remote site natural gas. Furthermore, the purity of ethylene produced by dehydrogenation of ethane is typically too low to be used as a feedstock in many downstream processes and ethylene is notoriously difficult to purify. Consequently, expensive purification processes are usually required.

What is needed is a method of improving the purity and reducing the transportation costs of ethylene produced from ethane.

### BRIEF SUMMARY OF PREFERRED EMBODIMENTS

Some of the preferred embodiments of the present invention relate to methods for sequentially converting ethane to ethylene and then to ethanol. Preferably, the ethane is converted to ethylene using catalytic oxidative dehydrogenation and the ethylene is converted to ethanol by direct catalytic hydration. According to some preferred embodiments, the ethanol is purified and/or transported.

Some of the preferred embodiments of the present invention relate to methods for reducing ethylene transportation and/or purification costs by converting ethylene to ethanol, transporting and/or purifying the ethanol, and converting the ethanol back to ethylene. According to some preferred embodiments, the ethylene is produced by dehydrogenation of ethane derived from natural gas. Preferably, the conversion of ethane to ethylene and ethylene to ethanol occurs at a site near the natural gas reserves and the conversion of ethanol to ethylene occurs at the ethylene customer site.

Some of the preferred embodiments of the present invention relate to products prepared according to the methods described.

### BRIEF DESCRIPTION OF THE DRAWINGS

For a more detailed description of the present invention, reference will now be made to the accompanying drawings, wherein:

FIG. 1 depicts a simplified flow diagram for a multi-step process comprising a dehydrogenation/hydration reaction.

FIG. 2 depicts a block diagram schematic for a multi-step process comprising a dehydrogenation/hydration reaction.

FIG. 3 depicts a simplified flow diagram for a multi-step process comprising a hydration/ dehydration reaction.

FIG. 4 depicts a block diagram schematic for a multi-step process comprising a hydration/ dehydration reaction.

FIG. 5 depicts a simplified flow diagram for a multi-step process comprising a dehydrogenation/hydration/dehydration reaction.

### DETAILED DESCRIPTION OF PREFERRED EMBODIMENTS

The preferred embodiments of the present invention comprise various combinations of dehydrogenation, hydration and dehydration reactions. Some preferred embodiments derive from the conception of a novel method of producing ethanol from ethane using a combination of dehydrogenation and hydration reactions. Other preferred embodiments of the present invention derive from the conception of a novel technique for reducing ethylene transportation and/or purification costs by hydrating the ethylene to ethanol, purifying and/or transporting the ethanol, and then dehydrating the ethanol to ethylene.

Ethanol is a highly versatile chemical and is useful as a solvent, germicide, beverage, antifreeze, fuel, depressant and reactant in chemical syntheses. Common synthetic routes to ethanol include hydration from ethylene and fermentation of sugar, starch and cellulose. Thus, the typical synthetic starting materials for ethanol production are ethylene, sugar, starch and cellulose. Although commercially viable, the intrinsic cost of these techniques is fundamentally limited by the expense of the starting materials.

The synthesis of ethanol from ethane derived from natural gas offers a variety of benefits over prior art methods of preparing ethanol. First, the ethane in natural gas (particularly natural gas located at remote reserves) has a relatively low intrinsic cost when compared to the costs for ethylene, sugar, starch and cellulose. Thus, the ethane in such natural gas is an economically-attractive starting material for ethanol manufacture. Second, the transportation costs of ethanol are relatively low when compared to the transportation costs of ethane or ethylene. Thus, ethanol is an attractive product for remote site manufacturing. For both of these reasons, the production of ethanol from ethane in natural gas presents an attractive alternative to prior art methods.

Conversion of Ethane to Ethanol

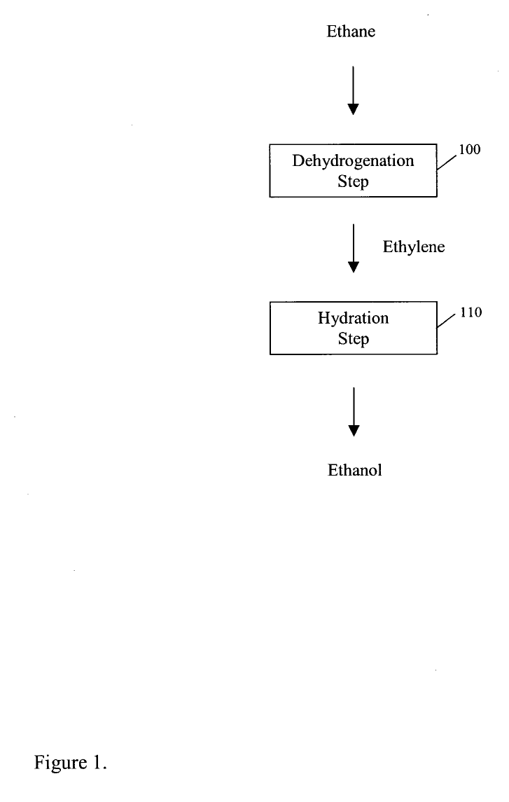

Some of the preferred embodiments of the present invention relate to processes for the conversion of ethane to ethanol through a multi-step process comprising a dehydrogenation/hydration reaction, depicted as a simplified flow diagram in FIG. 1. According to some of the preferred embodiments of the present invention, the process comprises a dehydrogenation step 100 in which ethane is converted to ethylene. The dehydrogenation process can be any type of process capable of yielding ethylene from ethane. Preferably, the dehydrogenation process is one of those disclosed below. More preferably, the dehydrogenation process is a catalytic oxidative dehydrogenation process performed in a short contact time reactor. The process also comprises a hydration step 110 in which ethylene is converted to ethanol. The hydration process can be any type of hydration process capable of yielding ethanol from ethylene. Preferably, the dehydrogenation is an embodiment disclosed below. More preferably, the hydration process is a direct catalytic hydration. The preferred dehydrogenation and hydration techniques are described in further detail below.

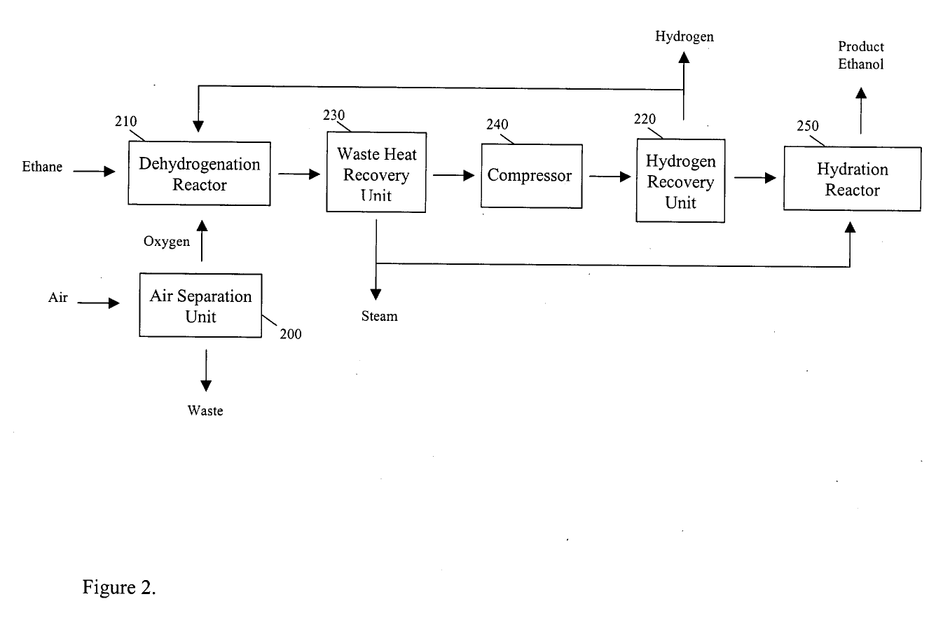

FIG. 2 depicts a block diagram schematic for a preferred embodiment of the present invention. Ethane from a natural gas stream and oxygen from an air separation unit (ASU) 200 are mixed and enter a dehydrogenation reactor 210 maintained under reaction promoting conditions. The ethane/oxygen feedstock can be supplemented by a hydrogen stream from hydrogen recovery unit 220, which is downstream of dehydrogenation reactor 210. The ethylene product stream from dehydrogenation reactor 210 then passes through waste heat recovery unit 230 and compressor 240, which recover waste heat and pressurize the ethylene product stream, respectively. The resulting cooled, pressurized ethylene product stream passes through hydrogen recovery unit 220, which separates hydrogen present in the ethylene product stream. Following hydrogen removal, the ethylene product stream is converted to ethanol in hydration reactor 250. As will be immediately evident to those of skill in the art, additional process steps—e.g., purification steps—are within the spirit and scope of the invention.

Purification and Transportation of Ethylene as Ethanol

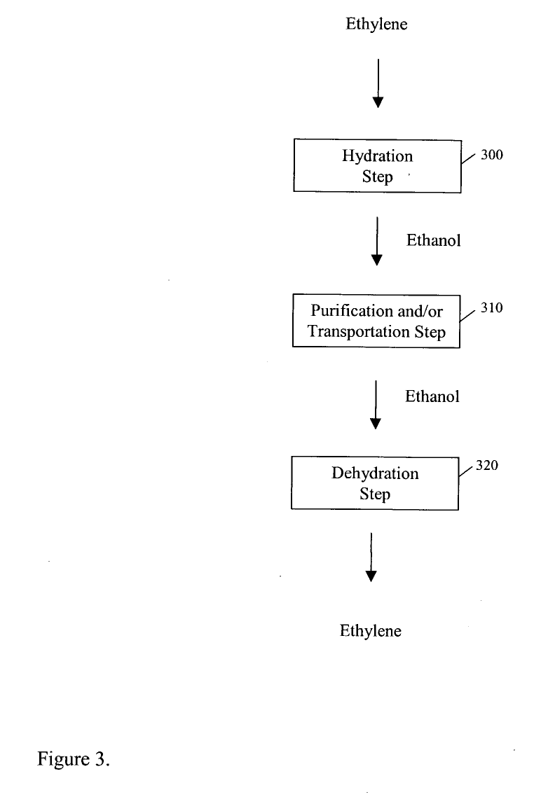

Some of the preferred embodiments of the present invention relate to processes for the conversion of ethylene to ethanol and back to ethylene through a multi-step process comprising a hydration/dehydration reaction, depicted as a simplified flow diagram in FIG. 3. According to some of the preferred embodiments of the present invention, the process comprises a hydration step 300 in which ethylene is converted to ethanol. The hydration process can be any type of hydration process capable of yielding ethanol from ethylene. Preferably, the hydration process is an embodiment disclosed below. More preferably, the hydration process is a direct catalytic hydration. The preferred process also comprises a purification and/or transportation step 310 in which the ethanol is purified and/or transported to another location. The process also comprises a dehydration step 320 in which ethanol is converted back to ethylene. The dehydration process can be any type of dehydration process capable of yielding ethylene from ethanol. Preferably, the dehydration process is an embodiment disclosed below. The preferred hydration and dehydration techniques are described in further detail below.

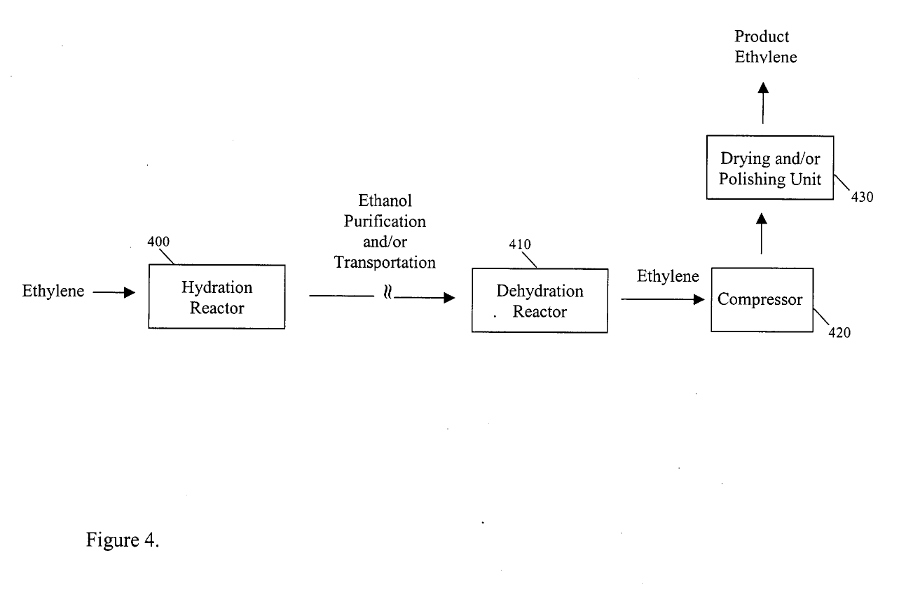

FIG. 4 depicts a block diagram schematic for a preferred embodiment of the present invention. Ethylene is converted to ethanol in hydration reactor 400. The ethylene is then treated—e.g., by purification and/or transportation—and converted back to ethylene in dehydration reactor 410. As used herein, the term “treated” or “treatment” refers to any process that may be advantageously performed on ethanol, including purification and transportation, to alter its quality or location. The ethylene product stream is then pressurized in compressor 420 and remaining impurities are removed in drying and/or polishing unit 430 to yield a clean, dry ethylene product.

Conversion of Ethane to Treated Ethylene Through Ethanol Intermediate

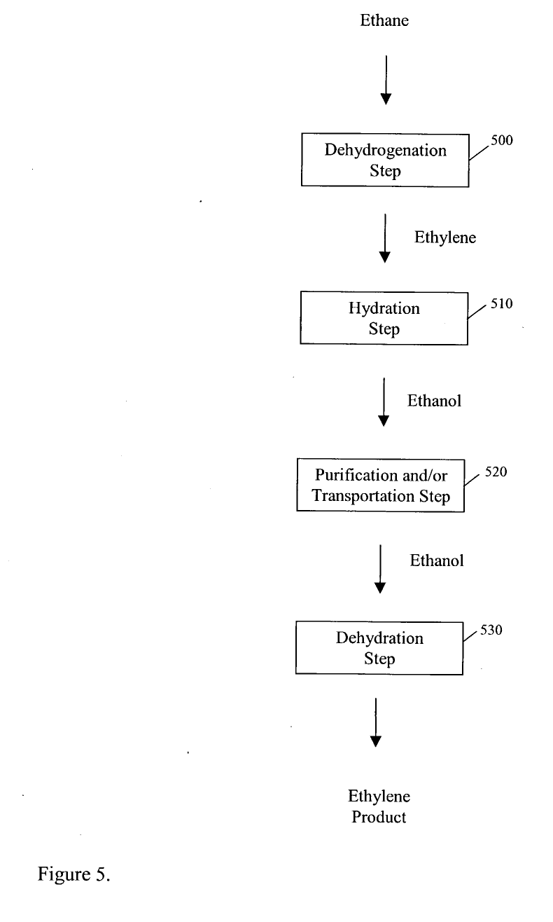

Some of the preferred embodiments of the present invention relate to processes for the conversion of ethane to ethylene through a multi-step process involving ethanol and comprising a dehydrogenation/hydration/dehydration reaction, depicted as a simplified flow diagram in FIG. 5. The process comprises a dehydrogenation step 500 in which ethane is converted to ethylene. Subsequently, a hydration step 510 is performed in which ethylene is converted to ethanol. The ethanol is then treated in purification and/or transportation step 520. The ethanol is then converted back to ethylene in dehydration step 530 to yield an ethylene product stream. As will be recognized by one of skill in the art, an appropriate block diagram can be constructed from FIGS. 2 and 4.

Preferred Methods for Dehydrogenation Reaction

Any acceptable process for converting ethane to ethylene may be used in the present invention. Exemplary methods for preparing ethylene from ethane include, but are not limited to, fluidized catalytic cracking, steam pyrolysis and catalytic ODH. Therefore, without limiting the scope of the invention, the preferred embodiments of the present invention employ a catalytic ODH process for the conversion of ethane to ethylene as depicted in reaction [2] above.

As used herein, the term “ODH catalyst” refers to the overall catalyst including, but not limited to, any base metal, promoter metal and refractory support. A variety of catalyst compositions are suitable as ODH catalysts. Without limiting the scope of the invention, the preferred embodiments employ a catalyst comprising a promoter metal and a base metal on a refractory support. Many promoter metals increase catalyst activity in ODH processes and are within the scope of the present invention. As an example, and without limiting the scope of the invention, promoter metals in ODH catalysts include Group VIII metals—i.e., platinum, rhodium, ruthenium, iridium, nickel, palladium, iron, cobalt and osmium. Platinum and palladium are the preferred promoter metals. However, as is evident to those of skill in the art, other promoter metals can also be used. Furthermore, a combination of promoter metals is also within the scope of the invention. Consequently, references herein to the promoter metal are not intended to limit the invention to one promoter metal.

As used herein, the term “promoter metal loading” refers to the percent by weight promoter metal in the ODH catalyst, measured as the weight of reduced promoter metal relative to the overall weight of the ODH catalyst. Preferably, the promoter metal loading is between about 0.005 and about 0.1 weight percent. The promoter metal loading is more preferably between about 0.005 and about 0.095, still more preferably between about 0.005 and about 0.075, and yet still more preferably between about 0.005 and about 0.05 weight percent.

Some of the preferred embodiments of the present invention employ one or more base metals in addition to the promoter metal. A variety of base metals exhibit catalytic activity in ODH processes and are within the scope of the present invention. As an example, and without limiting the scope of the invention, base metals useful in the preferred embodiments of the present invention include Group IB-IIB metals, Group IVB-VIIB metals, Group IIA-VA metals, lanthanide metals, scandium, yttrium, actinium, iron, cobalt, nickel, their oxides and combinations thereof. More preferably, the base metal is selected from the group consisting of manganese, chromium, tin, copper, gold, their corresponding oxides and combinations thereof. A combination of base metals is within the scope of the invention. Consequently, references herein to the base metal are not intended to limit the invention to one base metal.

As used herein, the term “base metal loading” refers to the percent by weight base metal in the ODH catalyst, measured as the weight of reduced base metal relative to the overall weight of the ODH catalyst. When present, the base metal is preferably present at a base metal loading of between about 0.5 and about 20 weight percent, more preferably between about 1 and about 12 weight percent, and still more preferably between about 2 and about 6 weight percent. The molar ratio of the optional base metal to the promoter metal is preferably about 10 or higher, more preferably about 15 or higher, still more preferably about 20 or higher, and yet still more preferably about 25 or higher.

Preferably, the promoter metal and the base metal, if present, are deposited on refractory supports configured as wire gauzes, porous monoliths, or particles. The term “monolith” refers to any singular piece of material of continuous manufacture such as solid pieces of metal or metal oxide or foam materials or honeycomb structures. Two or more such catalyst monoliths may be stacked in the catalyst zone of the reactor if desired. For example, the catalyst can be structured as, or supported on, a refractory oxide “honeycomb” straight channel extrudate or monolith, made of cordierite or mullitc, or other configuration having longitudinal channels or passageways permitting high space velocities with a minimal pressure drop. Such configurations are known in the art and described, for example, in Structured Catalysts and Reactors, A. Cybulski and J. A. Moulijn (Eds.), Marcel Dekker, Inc., 1998, p. 599-615 (Ch. 21, X. Xu and J. A. Moulijn, “Transformation of a Structured Carrier into Structured Catalyst”), which is hereby incorporated herein by reference.

Some preferred monolithic supports include partially stabilized zirconia (PSZ) foam (stabilized with Mg, Ca or Y), or foams of α-alumina, cordierite, titania, mullite, Zr-stabilized α-alumina, or mixtures thereof. A preferred laboratory-scale ceramic monolith support is a porous alumina foam with approximately 6,400 channels per square inch (80 pores per linear inch). Preferred foams for use in the preparation of the catalyst include those having from 30 to 150 pores per inch (12 to 60 pores per centimeter). The monolith can be cylindrical overall, with a diameter corresponding to the inside diameter of the reactor tube.

Alternatively, other refractory foam and non-foam monoliths may serve as satisfactory supports. The promoter metal precursor and any base metal precursor, with or without a ceramic oxide support forming component, may be extruded to prepare a three-dimensional form or structure such as a honeycomb, foam or other suitable tortuous-path structure.

More preferred catalyst geometries employ distinct or discrete particles. The terms “distinct” or “discrete” particles, as used herein, refer to supports in the form of divided materials such as granules, beads, pills, pellets, cylinders, trilobes, extrudates, spheres, other rounded shapes or another manufactured configuration. Alternatively, the divided material may be in the form of irregularly shaped particles. Preferably at least a majority—i.e., greater than about 50 percent of the particles or distinct structures have a maximum characteristic length (i.e., longest dimension) of less than six millimeters, preferably less than three millimeters. Preferably, these particulate-supported catalysts are prepared by impregnating or washcoating the promoter metal and base metal, if present, onto the refractory particulate support.

Numerous refractory materials may be used as supports in the present invention. Without limiting the scope of the invention, suitable refractory support materials include silicon carbide, boron carbide, tungsten carbide, silicon nitride, boron nitride, tungsten nitride, zirconia, magnesium stabilized zirconia, zirconia stabilized alumina, yttrium stabilized zirconia, calcium stabilized zirconia, alumina, cordierite, titania, silica, magnesia, niobia, vanadia, nitrides, silicon nitride, carbides, silicon carbide, cordierite, cordierite-alpha alumina, zircon mullite, spodumene, alumina-silica magnesia, zircon silicate, sillimanite, magnesium silicates, zircin, petalite, carbon black, calcium oxide, barium sulfate, silica-alumina, alumina-zirconia, alumina-chromia, alumina-ceria, and combinations thereof. Preferably, the refractory support comprises alumina, zirconia or combinations thereof. Alumina is preferably in the form of alpha-alumina (α-alumina); however, the other forms of alumina have also demonstrated satisfactory performance.

The promoter metal and base metal, when present, may be deposited in or on the refractory support by any method known in the art. Without limiting the scope of the invention, acceptable methods include incipient wetness impregnation, chemical vapor deposition, co-precipitation, and the like. Preferably, the base and promoter metals are deposited by the incipient wetness technique.

The preferred embodiments of the processes of the present invention employ an ethane feedstock and an oxidant feedstock that are mixed to yield a reactant mixture, which is sometimes referred to herein as the reactant gas mixture. The oxidant feedstock comprises an oxidant capable of oxidizing at least a portion of the ethane feedstock. Appropriate oxidants may include, but are not limited to, I₂, O₂, N₂O, CO₂ and SO₂. Use of the oxidant shifts the equilibrium of the dehydrogenation reaction toward complete conversion through the formation of compounds containing the abstracted hydrogen (e.g., H₂O and HI). Preferably, the oxidant comprises a molecular oxygen-containing gas. Without limiting the scope of the invention, representative examples of acceptable molecular oxygen-containing gas feedstocks include pure oxygen gas, air and O₂-enriched air.

As depicted in equation [4], the complete combustion of ethane requires a stoichiometrically predictable quantity of oxygen:

C₂H₆+7/2O₂→2CO₂+3H₂O [4].

According to equation 4, an atomic oxygen-to-carbon ratio of 7:2 represents the stoichiometric ratio for complete combustion of ethane. Preferably, the composition of the reactant gas mixture is such that the atomic oxygen-to-carbon ratio is between about 0.05:1 and about 5:1. In some embodiments, the reactant mixture may also comprise steam. Steam may be used to activate the catalyst, remove coke from the catalyst, or serve as a diluent for temperature control. The ratio of steam to carbon by weight, when steam is added, may preferably range from about 0 to about 1.

Preferably, a short contact time reactor (SCTR) is used. Use of a SCTR for the commercial scale conversion of ethane to ethylene allows reduced capital investment and increases ethylene production significantly. The preferred embodiments of the present invention employ a very fast contact (i.e., millisecond range)/fast quench (i.e., less than one second) reactor assembly such as those described in the literature. For example, co-owned U.S. Pat. No. 6,409,940 describes the use of a millisecond contact time reactor for use in the production of synthesis gas by catalytic partial oxidation of methane. The use of a similar reactor for ODH is described in a commonly-assigned currently-pending application entitled “Oxidative Dehydrogenation of Hydrocarbons Using Catalysts with Trace Catalytic Metal Loading,” Attorney Docket No. 1856-18900, application Ser. No. 10/266,404. The disclosures of these references are hereby incorporated herein by reference.

The ODH catalyst may be configured in the reactor in any arrangement including fixed bed, fluidized bed, or ebulliating bed (sometimes referred to as ebullating bed) arrangements. A fixed bed arrangement employs a stationary catalyst and a well-defined reaction volume whereas a fluidized bed utilizes mobile catalyst particles. Conventional fluidized beds include bubbling beds, turbulent fluidized beds, fast fluidized beds, concurrent pneumatic transport beds, and the like. A fluidized bed reactor system has the advantage of allowing continuous removal of catalyst from the reaction zone, with the withdrawn catalyst being replaced by fresh or regenerated catalyst. A disadvantage of fluidized beds is the necessity of downstream separation equipment to recover entrained catalyst particles. Preferably, the catalyst is retained in a fixed bed reaction regime in which the catalyst is retained within a well-defined reaction zone. Fixed bed reaction techniques are well known and have been described in the literature. Irrespective of catalyst arrangement, the reactant mixture is contacted with the catalyst in a reaction zone while maintaining reaction promoting conditions.

The reactant gas mixture is heated prior to or as it passes over the catalyst such that the reaction initiates. In accordance with one preferred embodiment of the present invention, a method for the production of ethylene includes contacting a preheated reactant gas mixture with a catalyst containing a Group VIII metal and a refractory support sufficient to initiate oxidative dehydrogenation, maintaining a contact time of the reactant gas mixture with the catalyst for less than about 30 milliseconds, and maintaining oxidative dehydrogenation promoting conditions. Preferably, the ODH catalyst composition and the reactant gas mixture composition are such that oxidative dehydrogenation promoting conditions can be maintained with a preheat temperature of about 600° C. or less. More preferably, the ODH catalyst composition and the reactant mixture composition are such that oxidative dehydrogenation promoting conditions can be maintained with a preheat temperature of about 300° C. or less.

Reaction productivity, conversion and selectivity are affected by a variety of processing conditions including temperature, pressure, gas hourly space velocity (GHSV) and catalyst arrangement within the reactor. As used herein, the term “maintaining reaction promoting conditions” refers to controlling these reaction parameters, as well as reactant mixture composition and catalyst composition, in a manner in which the desired ODH process is favored.

The reactant gas mixture may be passed over the catalyst in any of a wide range of gas hourly space velocities. Gas hourly space velocity (GHSV) is defined as the volume of reactant gas per volume of catalyst per unit time. Although for ease in comparison with prior art systems space velocities at standard conditions have been used to describe the present invention, it is well recognized in the art that residence time is inversely related to space velocity and that high space velocities correspond to low residence times on the catalyst and vice versa. High throughput systems typically employ high GHSV and low residence times on the catalyst.

Preferably, GHSV for the present process, stated as normal liters of gas per liters of catalyst per hour, ranges from about 20,000 to about 200,000,000 hr⁻¹, more preferably from about 50,000 to about 50,000,000 hr⁻¹. The GHSV is preferably controlled so as to maintain a reactor residence time of no more than about 30 milliseconds for the reactant gas mixture. An effluent stream of product gases including ethylene, unconverted ethane, H₂O and possibly CO, CO₂, H₂ and other byproducts exits the reactor. In a preferred embodiment, the ethane conversion is at least about 40 percent and the ethylene selectivity is at least about 30 percent. More preferably, the ethane conversion is at least about 60 percent and the ethylene selectivity is at least about 50 percent. Still more preferably, the ethane conversion is at least about 80 percent and the ethylene selectivity is at least about 55 percent. Still yet more preferably, the ethane conversion is at least about 85 percent and the ethylene selectivity is at least about 60 percent.

Hydrocarbon processing techniques typically employ elevated temperatures to achieve reaction promoting conditions. According to some preferred embodiments of the present invention, the step of maintaining reaction promoting conditions includes preheating the reactant mixture to a temperature between about 30° C. and about 750° C., more preferably not more than about 600° C. The ODH process typically occurs at temperatures of from about 450° C. to about 2,000° C., more preferably from about 700° C. to about 1,200° C. As used herein, the terms “autothermal,” “adiabatic” and “self-sustaining” mean that after initiation of the hydrocarbon processing reaction, additional or external heat need not be supplied to the catalyst in order for the production of reaction products to continue. Under autothermal or self-sustaining reaction conditions, exothermic reactions provide the heat for endothermic reactions, if any. Consequently, under autothermal process conditions, an external heat source is generally not required.

Hydrocarbon processing techniques frequently employ atmospheric or above atmospheric pressures to maintain reaction promoting conditions. Some embodiments of the present invention entail maintaining the reactant gas mixture at atmospheric or near-atmospheric pressures of approximately 1 atmosphere while contacting the catalyst. Advantageously, certain preferred embodiments of the process are operated at above atmospheric pressure to maintain reaction promoting conditions. Some preferred embodiments of the present invention employ pressures up to about 32,000 kPa (about 320 atmospheres), more preferably between about 200 and about 10,000 kPa (between about 2 and about 100 atmospheres).

Preferred Methods for Hydration Reaction

Any acceptable process for converting ethylene to ethanol may be used in the present invention. Exemplary methods have been previously described in, for example, K. Weissermel, Industrial Organic Chemistry (3d Ed., 1999) pp. 191-196 and J. E. Logsdon, Ethanol, Kirk—Othmer Encyclopedia of Chemical Technology, Fourth Edition, Volume 9, 812-860, 1994, which are hereby incorporated by reference. Therefore, without limiting the scope of the invention, the preferred embodiments of the present invention employ two well known routes for hydrating ethylene to yield ethanol.

First, ethanol may be formed from ethylene in a two-step indirect process employing concentrated H₂SO₄ and water. According to this process, ethylene is initially reacted with concentrated H₂SO₄ to yield a sulfuric acid ester as depicted in reaction [5]:

C₂H₄+H₂SO₄→C₂H₅OSO₃H [5].

Typical process temperatures are between about 55 and about 80° C. and typical process pressures are between about 10 and about 35 bar.

The sulfuric acid ester is then hydrolyzed with water according to reaction [6], yielding ethanol and regenerated H₂SO₄:

C₂H₅OSO₃H+H₂O→C₂H₅OH+H₂SO₄ [6].

Typical process temperatures for the hydration step are between about 70 and about 100° C. Thus, the net reaction is to hydrate ethylene to ethanol as depicted in reaction [7]:

C₂H₄+H₂O→C₂H₅OH [7].

More preferably, the ethanol is prepared from ethylene by direct catalytic hydration. This reversible reaction is typically carried out in the gas phase over acidic catalysts as depicted in reaction [8]:

$${C}_{2} {H}_{4} + {H}_{2} O {⇄}^{[ {H}^{+} ]} {C}_{2} {H}_{5} OH . \qquad [ 8 ]$$

Although a number of acid catalysts may be used, H₃PO₄/SiO₂ catalysts are particularly useful in this process. Typical process temperatures are between about 200 and about 400° C. and typical process pressures are between about 40 and about 100 bar. Because single-pass ethanol yields are typically equilibrium limited in the direct catalytic hydration process, unconverted ethylene is preferably recycled to increase yield.

Preferred Methods for Dehydration Reaction

Some of the preferred embodiments of the present invention employ the additional process of converting ethanol to ethylene. Any acceptable process for converting ethanol to ethylene may be used in the present invention. Therefore, without limiting the scope of the invention, the preferred embodiments employ a dehydration process, as depicted in reaction [9]:

C₂H₅OH→C₂H₄+H₂O [9].

Several of the processing considerations and reactor designs relevant to ethanol dehydration to ethylene have been described in U. Tsao and J. W. Reilly, Dehydrate Ethanol to Ethylene, Hydrocarbon Processing, 1978, pp. 133-136, which is hereby incorporated by reference in its entirety herein. The dehydration process may be performed using any acceptable reactor configuration. Preferably, the reactor is a fixed or fluidized bed catalytic reactor.

The catalytic dehydration of ethanol is conventionally believed to proceed through a two-step mechanism, as depicted in reactions [10] and [11]:

2C₂H₅OH→(C₂H₅)₂O+H₂O [10]

(C₂H₅)₂O→2C₂H₄+H₂ [11].

Temperature is a critical operating parameter. Excessive temperatures result in the formation of aldehydes whereas lower temperatures result in ether product. Consequently, reactor design should eliminate cold and hot spots. The catalyst is typically regenerated every few weeks to remove carbon deposits.

Any catalyst capable of facilitating the conversion of ethanol to ethylene is within the scope of the present invention. Therefore, without limiting the scope of the invention, suitable catalysts include aluminas, silica-aluminas, metallic oxides, promoted aluminas and supported phosphoric acid catalysts—e.g., phosphoric acid on coke. Preferably, the catalyst is either an activated alumina or a supported phosphoric acid catalyst.

The following commonly assigned application concurrently filed herewith is hereby incorporated herein by reference: “Method for Treating Alkanes”, Attorney Docket No. 1856-30500, application Ser. No. ______, filed concurrently herewith. Should the disclosure of any of the patents, patent applications, and publications that are incorporated herein conflict with the present specification to the extent that it might render a term unclear, the present specification shall take precedence.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention.

Accordingly, the scope of protection is not limited by the description set out above, but is only limited by the claims which follow, that scope including all equivalents of the subject matter of the claims. Each and every claim is incorporated into the specification as an embodiment of the present invention. Thus the claims are a further description and are an addition to the preferred embodiments of the present invention. Use of the term “optionally” with respect to any element of a claim is intended to mean that the subject element is required, or alternatively, is not required. Both alternatives are intended to be within the scope of the claim. The discussion of a reference in the Description of Related Art is not an admission that it is prior art to the present invention, especially any reference that may have a publication date after the priority date of this application. The disclosures of all patents, patent applications and publications cited herein are hereby incorporated herein by reference, to the extent that they provide exemplary, procedural or other details supplementary to those set forth herein.

What is claimed is:

1. A method for the production of ethanol from ethane comprising:
a) converting at least a portion of a reactant stream comprising ethane to an intermediate product stream comprising ethylene; and
b) converting at least a portion of the intermediate product stream comprising ethylene to a product stream comprising ethanol.

2. The method of claim 1 wherein step a) comprises a catalytic oxidative dehydrogenation reaction.

3. The method of claim 2 wherein the catalytic oxidative dehydrogenation reaction occurs in a short contact time reactor at a gas-hourly space velocity between about 20,000 and about 200,000,000 hr⁻¹.

4. The method of claim 1 wherein the ethane in the reactant stream derives at least in part from a source of natural gas.

5. The method of claim 1 wherein step b) comprises a hydration reaction.

6. The method of claim 5 wherein the hydration reaction comprises a direct catalytic hydration reaction.

7. A method for the treatment of ethylene comprising
a) converting at least a portion of a reactant stream comprising ethylene to an intermediate product stream comprising ethanol;
b) treating the intermediate product stream comprising ethanol; and
c) converting at least a portion of the intermediate product stream comprising ethanol to a product stream comprising ethylene.

8. The method of claim 7 wherein step a) comprises a hydration reaction.

9. The method of claim 8 wherein the hydration reaction comprises a direct catalytic hydration reaction.

10. The method of claim 7 wherein step b) comprises purification of the intermediate product stream comprising ethanol.

11. The method of claim 7 wherein step b) comprises transportation of the intermediate product stream comprising ethanol.

12. The method of claim 7 wherein step c) comprises a dehydration reaction.

13. A method for the production of ethylene from ethane comprising
a) converting at least a portion of a reactant stream comprising ethane to an intermediate product stream comprising ethylene;
b) converting at least a portion of the intermediate product stream comprising ethylene to an intermediate product stream comprising ethanol;
c) treating the intermediate product stream comprising ethanol; and
d) converting at least a portion of the intermediate product stream comprising ethanol to a product stream comprising ethylene.

14. The method of claim 13 wherein step a) comprises a catalytic oxidative dehydrogenation reaction.

15. The method of claim 14 wherein the catalytic oxidative dehydrogenation reaction occurs in a short contact time reactor at a gas-hourly space velocity between about 20,000 and about 200,000,000 hr⁻¹.

16. The method of claim 13 wherein the ethane in the reactant stream derives at least in part from a source of natural gas.

17. The method of claim 13 wherein step b) comprises a hydration reaction.

18. The method of claim 17 wherein the hydration reaction comprises a direct catalytic hydration reaction.

19. The method of claim 13 wherein step c) comprises purification of the intermediate product stream comprising ethanol.

20. The method of claim 13 wherein step c) comprises transportation of the intermediate product stream comprising ethanol.

21. The method of claim 13 wherein step d) comprises a dehydration reaction.

22. A product comprising ethanol prepared according to the method of claim 1.

23. A product comprising ethylene prepared according to the method of claim 7.

24. A product comprising ethylene prepared according to the method of claim 13.
